SEQUENCE LISTING (5 sequences)

SEQ ID NO: 1 - protein, 128 aa, Lama glama
DVQLVESGGGSVQGAAGGSLRLSCAASDITYSTDCMGWFRQAPGKEREGVATINNGRAITYYADSVKGRFTISQDNAKNTVYLQMNSLRPKDTAIYYCAARLRAGYCYPADYSMDYWGKGTQVTVSSG

SEQ ID NO: 2 - protein, 126 aa, Lama glama
DVQLEESGGGSVQTGGSLRLSCAASGYTYSSACMGWFRQGPGKEREAVADVNTGGRRTYYADSVKGRFTISQDNTKDMRYLQMNNLKPEDTATYYCATGPRRRDYGLGPCDYNYWGQGTQVTVSSG

SEQ ID NO: 3 - protein, 132 aa, Lama glama
MAQVQLQESGGGSVQDGGSLRLSCAASGYAYDTYYMGWFRQAPGKEREWVAGITSLVSGVAYYKYYTDSVKGRFTIFRDDDKNTVDLQMNSLKPEDTAIYYCAASRSGLRARLLRPELYEYWGQGTQVTVSS

SEQ ID NO: 4 - protein, 129 aa, Lama glama
MAQVQLQESGGGSVQAGGSLRLSCVASGDTYSSACMGWFRQAPGKEREGVATICTSTSMRTRYYADAVKARFTISQDNAKNTVYLQMNSLKPEDIAMYYCATGHTVGSSWRDPGAWRYWGQGTQVTVSS

SEQ ID NO: 5 - protein, 138 aa, Lama glama
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSLEHHHHHH

(19) United States
(12) Patent Application Publication
(10) Pub. No.: US 2019/0338044 A1
(43) Pub. Date: Nov. 7, 2019

(54) NANOBODY VAGINAL ADMINISTRATION SYSTEM AND PREPARATION METHOD AND USE THEREOF

(71) Applicant: Nanolattix Biotechnology Co. Ltd., Taiyuan, Shanxi (CN)

(72) Inventors: Zhican Qu, Taiyuan, Shanxi (CN); Shaoping LI, Taiyuan, Shanxi (CN)

(21) Appl. No.: 16/068,666

(22) PCT Filed: Sep. 8, 2017
(86) PCT No.: PCT/CN2017/101026

§ 371 Date: May 22, 2019

(30) Foreign Application Priority Data
Sep. 3, 2016 (CN) 201610800512.5

(51) Int. Cl. C07K 16/32; A61K 47/32; A61K 47/18; A61K 47/10; A61K 47/24; A61K 47/38; A61K 47/42; C07K 16/28; C07K 16/22; C07K 16/14

(52) U.S. Cl. CPC C07K 16/32; A61K 47/32; A61K 47/183; A61K 47/10; A61K 47/24; A61K 47/38; A61K 2039/541; C07K 16/2863; C07K 16/22; C07K 16/2866; C07K 16/14; C07K 16/2878; C07K 2317/24; A61K 47/42

(57) ABSTRACT

The invention discloses a scheme for preparing some specific nanobody system and delivering nanobody medicine through woman's vagina as well as its preparation method and applications. Women's vagina is rich of capillaries and lymphatic vessels, vaginal permeability is greater than the rectum, mouth, and skin. Vaginal administration can not only local medication, but also systemic medication. Due to the unstable activity of traditional antibody or high molecular protein, large volume and other reasons, the biomedical administration mode is basically limited to the injection type. Nanobodies are the smallest units known to bind target antigens. The three-dimensional structure of nanobodies determines its relative stable biological activity, also nanobody has the characteristics of high temperature resistance, acid-base resistance, good water-solubility and strong tissue penetration. Thus, a wide range of applications of the Nanobody delivery system in disease treatment and women's health care can be achieved by optimizing carrier formulation to maintain and enhance the stability and tissue penetration of the nanobody, Vaginal nanobody formulations include: creams, crosslinked polyvinyl alcohol (PEG) hydrophilic gels, vaginal gels, vaginal capsules and tablets, vaginal suppositories, vaginal rings, and vaginal film. Nanobody vaginal administration can be used for specific nanobody in the local application, also can be used to penetrate the vaginal mucosa into the bloodstream to reach the preset lesion site to play a role. The vaginal delivery system includes specific nanobodies directly against different target sites of disease, such as local targets include: vaginal infections, endometriosis, uterine fibroids, and contraception, while systemic targets include inflammation and Rheumatism, cancer, viral bacteria, cardiovascular diseases, diabetes mellitus, Alzheimer's disease, but the target sites of the vaginal administration of the nanobody is not limited thereto. The composition for vaginal administration of the nanobody includes, but not limited to, soluble macromolecular glycogel matrix, polyethylene glycol (PEG), levoglucose, polyamino acid, glycerin, and stirring and mixing with optimized formulation of hydrolase inhibitor. The invention discloses a preparation method and application of a nanobody vaginal delivery system, a safe and effective administration model, develops a new form of nanobody biomedicine and has a wide application prospect.

The present invention involves a vaginal administration system for specific nanobody biomedicine and the preparation of the vaginal administration system. The proposed nanobody vaginal drug delivery system combines biologically active specific nanobody drugs with carriers that can maintain the stability of the nanobody drug while facilitating the process of drug tissue penetration.

### TECHNICAL FIELD

The present invention belongs to the field of biological medicine technology and relates to a vaginal administration system for specific nano-antibody biomedicine and the preparation thereof, wherein the system can be utilized in the areas of disease treatment and health care.

### BACKGROUND

The vagina is a flexible tubular organ composed of mucosa and muscle tissue. It is rich in capillaries and lymphatic vessels, and without clear nerve endings. The permeability of the vagina is greater than the rectum, mouth, and skin. There is little pain with vaginal administration, and it is an effective drug release site for specific diseases and drugs, making it a promising non-invasive route of administration, thereby exerting local and systemic effects of the drug. Vaginal administration can be used not only locally, but also systemically. Vaginal administration has unique advantages in use and treatment. Vaginal administration can bypass the liver's first pass effect, high bioavailability, suitable for some drugs with severe gastrointestinal reactions, and can avoid peaks caused by multiple doses.

The vaginal local administration route has been recognized for a long time, and can be effectively used for treating infection, endometriosis, uterine fibroids, contraception, and induction of labor when exerted locally. Later, it was discovered that many drugs are absorbed into the systemic blood circulation through the vaginal mucosa such as proteins, peptide drugs and vaccines can be administered. This enables the development of a systemic vaginal drug delivery system.

There are many types of vaginal drug delivery systems at home and abroad.

Suppository vaginal drug delivery system: The vaginal suppository is solid at room temperature, and can be rapidly softened, melted or dissolved in the secretion liquid after being incorporated into the vaginal lumen, and gradually releases the drug to produce a local or systemic effect. The shapes of the vaginal suppository are spherical, egg-shaped, duck-billed, etc., weighing about 2 to 5 g, and having a diameter of 115 to 215 cm. The duckbill type is more common.

Tablet-type vaginal drug delivery system: compared with other types, vaginal tablets have more advantages: 1) convenient use, stable quality, high degree of mechanization; 2) Compared with the suppository, it overcomes the loss of the suppository matrix receptor temperature after melting together with the drug, affecting the curative effect, contaminating the clothing and the patient's discomfort; 3), vaginal effervescent tablets can quickly disintegrate and increase the distribution of drugs in the vagina, fast onset, high efficacy; 4), the bioadhesive vaginal tablet improves the anti-discharge ability of the preparation, prolongs the residence time in the vagina, and is beneficial to improve bioavailability.

Vaginal ring delivery system: The time and release of the vaginal ring are related to the drug content, wall thickness, structure and surface area, and the type of drug used and the interaction of the drug with the silicone rubber matrix. At present, there are mainly compound estrogen and progesterone vaginal ring for contraception, estrogen vaginal ring for hormone replacement therapy, danazol vaginal ring for endometriosis, immune vaginal ring for enhancing local immunity and preventing sexually transmitted diseases. A sustained release vaginal ring for the prevention of HIV infection.

Vaginal Capsule Drug Delivery System: Compared with ointments and suppositories, vaginal capsules have an attractive appearance, are comfortable and convenient to clean and hygienic when used, and the drugs are dispersed in a particulate state, and the dosage is accurate.

Vaginal gel delivery system: The gel applied to intravaginal administration is mainly a functional hydrogel, and the main materials are polymers having mucoadhesive force, such as natural cationic high polymer and polyacrylic anion high. Polymer, etc. Mucoadhesive hydrogels are weakly crosslinked polymers that swell in contact with water and spread over the surface of the mucosa. They adhere to the membrane at the site of absorption, positioning the drug delivery system at a location and prolonging the residence time.

Vaginal membrane drug delivery system: The membrane agent has the characteristics of close contact with the mucous membrane, large administration area and stable sustained release of the drug. The drug film can be directly covered in the vaginal mucosa and slowly released to achieve an effective drug concentration for a certain period of time. There are many studies on membrane agents in China, and they are used in contraception, termination of early pregnancy, postmenopausal vaginal diseases, vaginitis and so on.

In addition, the types of the vaginal administration system includes effervescent granules, powders, creams, lotions, and etc.

In short, vaginal absorption of drugs involves two important steps: the drug dissolves in the vagina and the drug passes through the vaginal membrane. Any physiological or formulation factors that affect drug dissolution and drug membrane transport can affect the absorption of the drug in the vagina. The physicochemical properties of the drug, including molecular weight, lipophilicity and ionic state, can affect the absorption of the drug in the vagina. It is currently accepted that small molecular weight lipophilic drugs are more easily absorbed. In addition, the drug must be sufficiently lipophilic to pass through the lipid continuous membrane or vaginal mucosa in a diffuse form, but a degree of water solubility is also required to ensure solubility in the vaginal fluid.

In 1993, Belgian scientists first reported that about half of the antibodies in the blood of camels had no light chain, and these “heavy-chain antibodies” (HCAbs) lacking the light chain were as close to targets as antigens like normal antibodies. When combined, they do not stick to each other or gather together like scFv.

The camel single-chain antibody comprises only one variable domain of heavy chain of HCAb (VHH) and two conventional constant CH2 and CH3 regions; importantly, the VHH region can be cloned and expressed separately. It has good structural stability and antigen-binding activity. VHH is the smallest unit known to bind to the target antigen, so VHH is also called Nanobody. Camel single-chain antibodies are characterized by high affinity and high specificity, while immunogenicity (although non-human, but low in immunogenicity) and toxicity are very low and do not easily adhere.

Being compared with the heavy chain variable region VH of the human antibody, the CDR3 of the cluster complementary region of the Nanobody is longer, can form a convex loop structure, can penetrate the antigen so as to better bind the antigen, and can thereby exhibit a higher affinity. In addition, the hydrophobic residue of the Nanobody is substituted with a hydrophilic residue, which is more water-soluble and less likely to form aggregates.

Nanobodies are the smallest units currently known to bind to a target antigen. The crystal size of VHH is 2.5 nm×4 nm, the molecular weight is only 12˜15KD, its molecular structure is relatively stable, it can withstand high temperature and maintain its activity in extremely harsh environments. Studies have shown that VHH remains 80% biologically active at 37° C. for one week, indicating that Nanobodies are fairly stable at room temperature, making them easier to store and transport than conventional antibodies. Nanobodies have strong and fast tissue penetration ability, which facilitates their entry into dense tissues such as solid tumors, and can effectively penetrate the vaginal mucosa, providing a new method for vaginal administration.

At the same time, the nanobody has reversible refolding ability, that is, easy to refold. The test shows that the nanobody still maintains high activity after being treated at a high temperature of 90° C. and can regain antigen binding ability. While all conventional antibodies lost activity after treatment at 90° C., irreversible polymerization occurred. Under harsh conditions, such as chaotropic agents, presence of proteases, and extreme pH denaturation, normal antibodies fail or decompose, while Nanobodies remain highly stable.

In addition, the nano-body also exhibits the characteristics of being difficult to be denatured or easily renatured after denaturation under the condition of a strong denaturing agent.

Compared with the conventional antibody, 1), Nano-antibodies are readily available and can be obtained by immunization, B lymphocyte isolation, and antibody library display technology screening; 2) It has good stability, and its internal fold contains multiple disulfide bonds, which makes it have good stability and can be placed at room temperature; 3), High solubility, not as easy to aggregate as scFv, nanobody is hydrophilic, has good water solubility, can improve the utilization rate of drugs; 4), good absorption, because of high solubility, the nano-antibody has the advantage of high absorption rate; 5), Nano-antibodies are easy to express, unlike traditional antibodies, which must be expressed in mammalian cells. It is difficult, low-yield, and cost-effective. VHH can be highly expressed in prokaryotic cells, and researchers have increased the yield to 2.5 g/L.; 6), Humanization is simple, and the homology with human heavy chain genes is 80-90%. Humanization has been successful; 7) Nano-antibodies easily cross the biofilm system and are easily coupled to other molecules.

With the continuous development of bioengineering technology, a large number of nano-body drugs are emerging. At present, the main types of its clinical trials are injections and oral agents. Because nano-body drugs have small molecular weight, poor stability in vivo, and are susceptible to gastrointestinal enzyme degradation and first-pass effects of liver enzyme system when administered orally, in order to achieve effective drug treatment concentration, patients need to repeat injection or oral administration for many times.

A vaginal delivery system refers to a controlled release drug transdermal delivery system that promotes the delivery of a therapeutic amount of a drug through the vaginal mucosa into the systemic circulation. It can avoid gastrointestinal absorption difficulties caused by the interaction of gastrointestinal pH, enzymes, food and other drugs, and avoid first-pass effects. It can also avoid the inconvenience caused by injection administration, prolong the curative effect after single administration, and control the treatment time of short half-life drugs through drug depot and controlled release characteristics.

The vaginal administration of Nanobodies is a creative innovation that is suitable for the characteristics of Nanobodies. Transvaginal administration of Nanobodies is a safe and effective method of administration, and the amount of proteolytic enzymes in the epidermal tissue is small, which is conducive to maintaining the stability of such drugs. The nano-antibody drug has small molecular weight, small volume, stable structure and biological activity, easy to pass through the vaginal mucosa and cortical tissue, achieves an ideal transdermal absorption effect, enters the blood circulation system, reaches a target of a predetermined disease, and exerts a drug effect.

For the transvaginal drug delivery system of Nanobody, no related reports have been reported at home or abroad.

### Technical Problems

### Problem Solution

### Technical Solution

The object of the present invention is to provide a nano-body vaginal drug delivery system which is non-toxic, low-cost and convenient to use, and is particularly suitable for administration of a vaginal site in which a nano-body drug is mixed with other drugs.

Further, it is an object of the present invention to provide a preparation method and application of the above vaginal administration system.

The nanobody vaginal drug delivery system of the invention comprises a biologically active specific Nanobody, and an antibody drug carrier for increasing the stability and tissue penetration of the Nanobody, including, but not limited to, water solubility. a polymer bioglycemic base, polyvinyl alcohol, polyamino acid, glycerin, phospholipid, gelatin, sodium carboxymethylcellulose, collagen, hydrolase inhibitor, or a mixture of several in any ratio, Specific Nanobodies include both humanized and non-humanized antibody forms. The nano-antibody vaginal drug delivery system is different from the injection form and the oral dosage form and is administered by administering the active ingredient of the drug through the vaginal mucosa, and has the advantages of no pain, self-administration and treatment at any time, compared with the injection form. It is simple to use and has high drug delivery efficiency.

In the present invention, the antibody drug carrier is composed of a water-soluble polymer bio-glycemic matrix, a matrix material such as polyvinyl alcohol and water, and the antibody drug carrier has a large drug loading amount and includes a plurality of drugs. Good affinity for plant extracts and chemical drugs, sustained release of vaginal mucosa, good transdermal effect, breathable, high skin comfort, no skin irritation and allergic reaction, ideal for transvaginal mucosal drug delivery platform In combination with a variety of drugs, it is possible to prepare a variety of sustained release vaginal mucosal drug delivery systems.

Further, preferred antibody drug carriers of the invention are collagen, including collagen peptides, collagen and gelatin. Combined with collagen as a nano-body application, it can accelerate the healing of ulcer-induced erosion surface caused by inflammation, greatly shorten the time required for tissue repair, and can also be used in vaginal drug delivery systems to treat other diseases.

In the present invention, the specific Nanobody comprises an active Nanobody, a Nanobody fragment, or a multi-targeted Nanobody polymeric linker.

The Nanobody of the present invention may also be an expression and purification of a Nanobody, or an active Nanobody released by an active probiotic microorganism in a drug delivery system, a multi-targeted Nanobody polymer-linked linker, a Nanobody and a protein even Conjugated, a combination of a Nanobody and a drug for expression and secretion in the vagina and adjacent organs.

Specifically, in the above-mentioned nano-antibody polymeric linker, the same or different nano-antibodies may be polymerized, or the nano-antibody may be combined with albumin to prolong the half-life of the drug in the antibody drug carrier and the human body. The half-life of Nanobodies is not very long and measures are needed to extend their half-life in antibody drug carriers and in the vagina and in humans. The nano-antibody preparation administered by the vaginal mucosa of the invention can be greatly extended by the fusion of the nano-antibody with the albumin, the Fc fusion, the PEG-forming and the like, and the half-life of the nano-antibody in the antibody drug carrier and the vagina can be greatly prolonged.

In the nano-antibody vaginal drug delivery system of the present invention, the Nano-antibody is a specific Nano-antibody directed against different lesions, and the target of the lesion includes, but is not limited to, inflammation, rheumatism, cancer, viral bacteria; cardiovascular disease, diabetes, endocrine disorders, etc.

Further, specifically, the targets targeted by the nano-antibody include, but are not limited to, HER2 (human epidermal growth factor receptor 2 or HER2/neu), EGFR (epidermal growth factor receptor), VEGF (vascular endothelial Growth factor), VEGFR, EGFa (epidermal growth factor a), FGFb (epidermal growth factor b), PD-1, PD-L1, CTLA4, Sclerostin, Glucagon-like peptide 1, Glucagon-like peptide receptor, interferon IL-4. IL-5, IL-6, IL-9, IL-13, IL-17a, TNFa (Tumor Necrosis Factor a), TNFb (Tumor Necrosis Factor b), and vaginal yeast, bacteria, viral infection, and etc.

Further, the nanobody vaginal drug delivery system of the present invention may comprise one or more specific Nanobodies and may be combined with other drugs to form a composite vaginal drug delivery system.

The nano-antibody vaginal drug delivery system is prepared into a cream, a hydrophilic gel, a vaginal gel, a vaginal suppository, a vaginal tablet, a vaginal capsule, a vaginal ring, a vaginal film, an effervescent granule, and a powder. Various external preparations suitable for vaginal administration, such as a lotion or cream, which are dispersed in water, gel or cream and applied to the vagina to release active nano-antibodies or antibody fragments on the vaginal mucosa and the surface of the skin. The active Nanobody or antibody fragment can effectively cross the vaginal mucosa or epidermal layer.

Specific applications of the nanobody vaginal delivery system of the present invention include the absorption of the preparation through the vaginal mucosa and cortical tissue into the systemic blood circulation to reach a predetermined lesion site, and also includes the use of a formulation system for passage through the vaginal mucosa and cortical tissue. Absorption is used for local application areas.

The use of the topical application area includes treatment for vaginal and adjacent organ diseases including, but not limited to, vaginal infection and inflammation, endometriosis, uterine fibroids, infertility.

The nanobody vaginal drug delivery system of the present invention can be used for treating autoimmune diseases, including skin and body in vitro, such as local inflammation in the vagina and anus, blood diseases, orthopedic diseases and cancer.

The nano-antibody vaginal drug delivery system of the present invention can also be used as a special health care product for women, for example, a nano-antibody vaginal drug delivery system for regulating vaginal flora, preventing sexually transmitted diseases, contraception, and increasing maintenance of sexual intercourse and sensitive. Bacterial vaginitis is a mixed infection caused by dysregulation of normal flora in the vagina, but there are no inflammatory changes in clinical and pathological features. Mold vaginitis is caused by the disorder of Candida albicans in the vagina. Candida albicans is a group of bacteria that the human body has. Fungal vaginitis is a kind of bacterial vaginitis. The nano-antibody vaginal administration health care product preparation of the invention can effectively regulate the vaginal flora and enhance health.

### Benefits of the Invention

### Benefits

The present invention discloses for the first time a drug delivery system that utilizes specific Nanobodies to pass through a woman's vagina, and methods for its preparation and use. Women's vagina is rich in capillaries and lymphatic vessels, and the vaginal permeability is greater than the rectum, mouth and skin. Vaginal administration can be used not only locally, but also systemically. Due to the unstable activity and large volume of traditional antibody or protein macromolecules, the mode of administration of biomedicine is currently limited to the injection dosage form. Nano-bodies are the smallest units known to bind to target antigens, and their three-dimensional structure determines the biological properties of nano-bodies relative to their relative stability. The invention enhances the stability and tissue penetration of the nano-body by optimizing the carrier formulation and realizes the wide application of the nano-body vaginal drug delivery system in the field of disease treatment and women's health care. The nano-antibody vaginal drug delivery system of the invention is a safe and effective drug delivery mode and opens up a new dosage form of nano-body biomedicine, and its application prospect is very broad.

### INVENTION EMBODIMENT

### Embodiments of the Invention

The following examples illustrate specific embodiments of the invention. However, it should be noted that the following are merely examples for the application of the present invention. Numerous modifications and alternative compositions, methods, and systems can be devised by those skilled in the art without departing from the spirit and scope of the invention. The appended claims are intended to cover such modifications and arrangements. Thus, although the invention has been described in detail below, it is merely one of the details of the embodiments of the invention. A number of variations and modifications made without departing from the core of the present invention: Nanobody vaginal delivery system are intended to be within the scope of the present invention.

### Example 1

The purpose of the present embodiment is to provide a transvaginal mucosal sustained-release drug delivery system using a water-soluble polymer protein material as a main substrate, a preparation method of the drug delivery system, and application of the drug delivery system.

The drug delivery system is a transvaginal mucosal drug delivery system using a water-soluble high-molecular bio-sugar gum as a main antibody drug carrier. In the drug delivery system, an array of weights of a typical antibody drug carrier is: water-soluble 18 parts of macromolecular bio-sugar gum base, 5 parts of polyvinyl alcohol, 10 parts of polyamino acid, 10 parts of glycerin, 8 parts of phospholipid, 2.5 parts of gelatin, 1 part of sodium carboxymethyl cellulose, and 20 parts of water.

For different Nanobodies, the above antibody drug carriers can be adjusted accordingly to better maintain and increase the stability and tissue penetration of the Nanobody.

In the present embodiment, the vaginal mucosal sustained-release drug delivery system using the water-soluble polymer bio-glycolipid as the main antibody drug carrier can be prepared by the following method. According to the prescription ratio, the water-soluble polymer bio-glycosidic matrix and the poly-collection are weighed. Vinyl alcohol is added to an appropriate amount of water, heated and stirred in a 95° C. water bath for 45 min to completely dissolve. Polyamino acid, glycerol, phospholipid, gelatin, sodium carboxymethyl cellulose are sequentially added to the above solution according to the prescription ratio, 60-70° C. The mixture was heated and stirred for 15 minutes, and the added auxiliary materials were completely dissolved and uniformly mixed; according to the above prescription ratio. 0.1 part of the nano-antibody was weighed and added to the above solution, and stirred uniformly; that is, a sustained release system for transvaginal mucosa administration.

The above vaginal mucosal drug delivery system can be further processed into a cream, a cross-linked PEG hydrophilic gel, a vaginal gel, a vaginal suppository, a vaginal tablet, a vaginal capsule, a vaginal ring, a vaginal film, and etc.

The above vaginal mucosal drug delivery system is also processed into a dosage form such as effervescent granules, powders, creams, lotions, and etc.

### Example 2

This example uses collagen as an antibody drug carrier to prepare a nano-body vaginal drug delivery system related preparation.

The collagen may be a collagen peptide, collagen, or gelatin.

The collagen peptide used in the present embodiment can be obtained by enzymatic hydrolysis of gelatin, neo-gelatin, pig skin, cowhide, pig bone, bovine bone, pig Achilles or calf, and its relative molecular weight less than 100 kD.

The collagen used in the present embodiment can be obtained by enzymatic hydrolysis of pig skin, cowhide, pork bone, bovine bone, pig Achilles or calf, and its relative molecular weight is 250-300 kD.

In the nanobody vaginal administration system prepared in the present embodiment, the ratio by weight of the active ingredient of the nano-body to the collagen is 0.001 to 0.1:1, and the preferred ratio by weight is 0.1:5 to 50. Wherein, the weight ratio of the active ingredient of the nano-antibody to the collagen refers to the ratio of the weight of the solid matter in the final product.

The collagen used in the nano-body collagen vaginal administration system of the present embodiment may be a collagen peptide or a collagen alone or may be a mixed feed of collagen peptide and collagen, mainly because the properties of the two are similar, but the molecular weight is difference. Collagen is a spiral fibrous protein twisted to form three peptide chains. It is an important protein that constitutes connective tissue in animals. It is mainly hydrolyzed from raw materials such as pigskin, cowhide, beef Achilles tendon, pig Achilles tendon, pig bone and beef bone.

The collagen used in the present embodiment is generally enzymatically hydrolyzed by a single enzyme such as pepsin, papain or fig enzyme. It is mostly carried out at a low temperature (below 1° C.) to prevent protein denaturation without destroying the unique right-handed helix. The structural body only excises the non-collagen tail peptide site, and its relative molecular weight is 250-300 kD, and has a complete triple helix structure. The collagen is further hydrolyzed to obtain gelatin having a relative molecular weight of 100 to 200 kD, and the triple helix structure has been destroyed. The collagen peptide is a further enzymatic hydrolyzed product of gelatin, and its relative molecular weight is below 100 kD, most of which exists in the form of collagen oligopeptides, and the relative molecular weight is below 40 kD.

The collagen used in the present embodiment can also be directly obtained from enzymatic hydrolysis of gelatin, neo-gelatin and etc. The relative molecular weight of collagen and gelatin is large; water solubility is poor; viscosity is high; and the mechanical strength is large. Auxiliary drug formability is good; and the wound can be adhered; and under the action of collagenase, the oligopeptide or amino acid can be enzymatically involved in tissue growth. Collagen has a relatively small relative molecular weight, contains more collagen oligopeptides, and is highly water-soluble, and is easily absorbed by the body. Moreover, due to its high content of proline and hydroxyproline, it can directly participate in the body cells of the body, growing and providing raw materials for it. Collagen, gelatin and collagen peptides can promote wound healing and tissue repair in traditional Chinese medicine preparations.

The main raw materials of collagen in the present invention are from one or more of gelatin, donkey-hide gelatin, new donkey-hide gelatin, pigskin, cowhide, pig bone, bovine bone, pig achilles tendon or cattle achilles tendon, after thorough enzymolysis. The enzymolysis method may be enzymatic digestion with any one or combination of pepsin, trypsin, neutral protease, alkaline diterpene, bromelain, fig protease or papain, also can be completely modeled on human digestion and absorption process of biomimetic enzymatic method (Firstly, pepsin enzymolysis method was used to simulate human stomach digestive process and physicochemical parameters, and then enzymatic hydrolysis of pancreatic (protein) enzyme was carried out to simulate human intestinal digestion and absorption process to obtain low molecular oligopeptides substance).

The main preparation method of the collagen peptide used in the present embodiment is taking collagen raw materials, washing with water or salt solution, and removing impurities such as fat and residual meat, and adjusting acid and alkali to adjust appropriate pH value, low temperature (0 to 1)° C.) enzymatic hydrolysis for 24 to 72 hours, centrifugation, take the supernatant, adjust the appropriate pH value, add the desired protease to fully digest, take the supernatant, add neutral salt salting out, dialysis and impurity removal, and further purification.

The main raw material of the collagen used in the present embodiment is derived from any one or more of pig skin, cowhide, pork bone, bovine bone, pig Achilles or Achilles tendon, which is obtained by appropriate enzymatic hydrolysis and its relative molecular weight is 250˜300 kD. The enzymatic hydrolysis method may be a single enzyme enzymatic hydrolysis of any one of pepsin, trypsin, neutral protease, alkaline protease, bromelain, ficin or papain, or combination of enzymatic hydrolysis of any of enzymes above.

The main preparation method of the collagen used in the present embodiment is taking collagen raw materials, washing with water or salt solution, and removing impurities such as fat and residual meat, and adjusting acid and alkali to adjust appropriate pH value, low temperature (0 to 1° C.) enzymatic hydrolysis for 24 to 72 hours, centrifugation, take the supernatant, add neutral salt salting out, dialysis and impurity removal, and further purification.

In the preparation process of the collagen peptide or collagen in the present embodiment, the acids used are malic acid, citric acid, acetic acid, or any one or more of the HP value-adjustable synthetic acid-base water. The alkali is an artificially adjustable acid-base water with an adjustable HP value.

In the preparation process of the collagen peptide or collagen according to the present embodiment, the salt used for salting out may be any one or more of ammonium sulfate, magnesium sulfate, sodium sulfate, sodium chloride or sodium phosphate.

In the preparation process of the collagen peptide or collagen according to the embodiment, if the alkali used is an artificially synthesized acid-base water with an adjustable HP value, the salting-out process is not required, and the preparation process is non-toxic and non-polluting.

### Example 3

A Nanobody is administered via a vaginal mucosal drug delivery system, wherein the Nanobody in the drug delivery system can eliminate inflammatory factors such as IL-1 alpha (Interleukin 1 alpha) TNF-alpha, IL-8 (Interleukin 8).

### Example 4

A nanobody is administered through a vaginal mucosal drug delivery system, wherein the nanobody in the drug delivery system passes through a vaginal gel to eliminate infectious bacteria such as Propionibacterium acnes (P. acnes).

### Example 5

A nano-antibody transvaginal mucosal drug delivery system is a vaginal gel based on nano-antibody targeting RANKL, which can be applied near the vaginal mucosa for the treatment of osteoporosis.

### Example 6

A nano-antibody transvaginal mucosal drug delivery system is a vaginal gel based on anti-tumor nano-antibodies targeting EGFR, HER2, VEGFR2, c-Met, CXCR7, etc., and can be applied to vaginal mucosa, or form nanometers. The particles penetrate in the cancerous area and are used to treat cancer.

### Example 7

A nano-antibody transvaginal mucosal drug delivery system is a vaginal gel based on a trivalent Nanobody that specifically inhibits TNFR1, and is resistant to inflammatory diseases.

The nanobody vaginal drug delivery system of the present invention may further comprise an active microorganism. The antibody or antibody fragment can be expressed and/or secreted on the vaginal mucosa and vaginal skin surface.

The nanobody pharmaceutical preparation according to any one of the preceding claims, wherein the antibody is a VHH type or VNAR type heavy chain immunoglobulin or a fragment thereof, preferably derived from camelids, most preferably from llama heavy chain antibody or a fragment, or the antibody is a domain antibody (dAb) or a fragment of an immunoglobulin heavy or light chain.

The nano-antibody vaginal drug delivery system of the present invention can improve bioavailability, reduce dosage, reduce adverse reactions, improve drug treatment index, and increase clinical drug safety and formulation compliance. Therefore, the drug delivery system of the present invention has the superiority that the conventional drug delivery system cannot match. However, there is currently no application of the nanobody vaginal drug delivery system of the present invention in the world.

The Nanobody vaginal gel formulation is a weakly crosslinked polymer that swells upon contact with water and spreads over the surface of the mucosa. They adhere tightly to the membrane at the site of absorption, positioning the drug delivery system at a location and extending residence time. Although the vaginal epithelium is not a glandular cell that produces mucus, but it is infiltrated in a vaginal fluid containing cervical mucus so that adhesion of the mucous membrane can occur.

Such mucosal adhesion has three steps: 1) Affected by the surface energy effect and the expansion process; 2) The polymer chain passing through the polymer mucous membrane contact surface is unwound from the entangled state; 3) The exposed active site binds to the tissue macromolecule. The dry hydrogel sticks to the moist tissue and is quite strong. The water is absorbed from the surface of the tissue to dehydrate the surface and reduce the surface pressure, which is adsorbed on the mucosal surface like an anchor, thus prolonging the local action time of the drug.

Some of the Nanobody VHH sequences which can be carried out by the present invention are listed below, and the sequences have been reported. However, the Nanobody VHH sequence suitable for use in the present invention is not limited to this.

VHH-αHER2 sequence containing 128 amino acids 1:
DVQLVESGGG, SVQGAAGGSL, RLSCAASDIT, YSTDCMGWFR,
QAPGKEREGV, ATINNGRAIT, YYADSVKGRF, TISQDNAKNT,
VYLQMNSLRP, KDTAIYYCAA, RRLAGYCYPA, DYSMDYWGKG,
TQVTVSSG.
VHH-αHER2 sequence containing 126 amino acids 2:
DVQLEESGGG, SVQTGGSLRL, SCAASGYTYS, SACMGWFRQG,
PGKEREAVAD, VNTGGRRTYY, ADSVKGRFTI, SQDNTKDMRY,
LQMNNLKPED, TATYYCATGP, RRRDYGLGPC, DYNYWGQGTQ,
VTVSSG.
VHH-α VEGF sequence containing 132 amino acids 1:
MAQVQLQESG, GGSVQDGGSL, RLSCAASGYA, YDTYYMGWFR,
QAPGKEREWV, AGITSLVSGV, AYYKYYTDSV, KGRFTIFRDD,
DKNTVDLQMN, SLKPEDTAIY, YCAASRSGLR, ARLLRPELYE,
YWGQGTQVTV, SS.
VHH-α VEGF sequence 2 containing 129 amino acids:
MAQVQLQESG, GGSVQAGGSL, RLSCVASGDT, YSSACMGWFR,
QAPGKEREGV, ATICTSTSMR, TRYYADAVKA, RFTISQDNAK,
NTVYLQMNSL, KPEDIAMYYC, ATGHTVGSSW, RDPGAWRYWG,
QGTQVTVSS.
VHH-αEGFR sequence containing 138 amino acids:
QVQLQESGGG, LVQPGGSLRL, SCAASGRTFS, SYAMGWFRQA,
PGKQREFVAA, IRWSGGYTYY, TDSVKGRFTI, SRDNAKTTVY,
LQMNSLKPED, TAVYYCAATY, LSSDYSRYAL, PQRPLDYDYW,
GQGTQVTVSS, LEHHHHHH.

The above non-humanized Nanobody VHH sequence can be humanized by the amino acid sequence of the naturally occurring VHH sequence domain, and can be one or more amino acid residues in the amino acid sequence of the naturally occurring VHH sequence domain. The base is replaced by an amino acid residue present at the corresponding position in the conventional human VH sequence domain.

1. Nanobody vaginal drug delivery systems, combining biologically active specific Nanobodies drug with its carriers that can maintain nanobody stability while facilitating its tissue penetration, includes but not limited to water soluble high molecular weight bio-glucose a mixture of a matrix, polyvinyl alcohol, polyamino acid, glycerol, phospholipid, gelatin, sodium carboxymethylcellulose, collagen, hydrolase inhibitor, or a mixture of any of several, said specific Nanobody comprising Humanized and non-humanized nanobody forms.

2. The drug delivery system of claim 1 where the nanobody drug carrier is collagen.

3. The drug delivery system of claim 1 where said specific Nanobody comprises an active Nanobody, a Nanobody fragment, or a multi-targeted Nanobody polymeric linker.

4. The drug delivery system according to claim 3, where the nanobody polymerized linker polymerizes between the same or different Nanobodies, or the Nanobody binds to a protein, and the Nanobody Chemical small molecule drug combination.

5. The drug delivery system according to claim 1, where specific nanobody is an expressed and purified nanobody, or an active nanobody, a multi-targeted nanobody polymerized by an active probiotic microorganism in a drug delivery system. Linkers, conjugates of nanobodies and proteins, and conjugates of nanobodies and drugs for expression and secretion in the vagina and adjacent organs.

6. The drug delivery system according to claim 1, 2, 3, 4 or 5, where the Nanobody in the drug delivery system is a specific Nanobody against different lesions such as women's intravaginal environment and inflammation of adjacent cervical and ovarian organs and viral bacterial infections and tumors, including skin diseases, inflammation and rheumatism, cancer, viral bacteria, cardiovascular disease, diabetes, Alzheimer's disease, brain tumors, bone Osteoporosis, psoriasis, asthma, specific dermatitis, and chronic sinusitis.

7. The drug delivery system of claim 6, where the target of the Nanobody-targeted condition includes, but is not limited to, HER2, EGFR, VEGF, VEGFR, FGFa, FGFb, TNFa, TNFb, PD-1, PD-L1, CTLA4, Sclerostin, Glucagon-like peptide 1, Glucagon-like peptide receptor, interferon IL-4, IL-5, IL-6, IL-9, IL-13, IL-17a, vagina Yeast, bacteria, and virus infection.

8. The drug delivery system according to claim 1, 2, 3, 4 or 5, where the drug delivery system comprises one or more specific Nanobodies and is compatible with other drugs and combined to form a composite vaginal delivery system.

9. The drug delivery system according to claim 1, 2, 3, 4 or 5, where the type of the drug delivery system is a cream, hydrophilic gel, vaginal gel, vaginal suppository, vaginal tablets, vaginal capsules, vaginal ring, vaginal film, effervescent granules, powders, creams or lotions.

10. The use of the drug delivery system of claim 1 as a preparation for absorption through the vaginal mucosa and cortical tissue into the systemic blood circulation to a predetermined lesion site.

11. The use of the drug delivery system of claim 1 for the absorption of a formulation for use in a topical application area by vaginal mucosa and cortical tissue.

12. The use of the drug delivery system of claim 1 to prepare a formulation for the treatment of vaginal and adjacent organ diseases through vaginal mucosa and cortical tissue.

13. The use according to claim 12, where the diseases of the vagina and adjacent organs include, but are not limited to, vaginal infections and inflammation, endometriosis, uterine fibroids, infertility.

14. The use of the drug delivery system of claim 1 for the preparation of a feminine health care product.

15. The use of the drug delivery system of claim 1 to prepare a vaginal flora formulation.
